(19) United States
(12) Patent Application Publication
(10) Pub. No.: US 2007/0066606 A1
(43) Pub. Date: Mar. 22, 2007

(54) Benzylbenzimidazolyl derivatives

(76) Inventors: Wolfgang Stahle, Ingelheim (DE); Alfred Jonczyk, Darmstadt (DE); Wilfried Rautenberg, Reinheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/571,587

(22) PCT Filed: Aug. 17, 2004
(86) PCT No.: PCT/EP04/09205

§ 371 Date: Mar. 10, 2006

(30) Foreign Application Priority Data
Sep. 12, 2003 (DE) 103 42 503.9

(51) Int. Cl. A61K 31/541; A61K 31/5377; A61K 31/496; A61K 31/454; A61K 31/427; A61K 31/422; A61K 31/4184; C07D 417/02; C07D 413/02; C07D 403/02

(52) U.S. Cl. 514/227.8; 514/232.2; 514/254.06; 514/322; 514/365; 514/374; 514/394; 544/60; 544/139; 544/370; 546/199; 548/181; 548/215; 548/305.4; 548/307.4

(57) ABSTRACT

Novel benzyl-benzimidazolyl derivatives as inhibitors of tyrosine kinases, particularly TIE-2, VEGFR, PDGFR, FGFR and/or FLT/KDR, for the treatment of tumors, according to formula (I), wherein the radicals R¹, R², r and s are defined according to Claim (1).

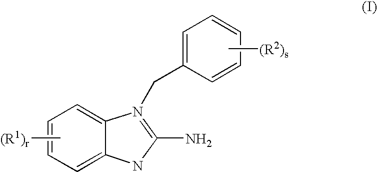

### BACKGROUND OF THE INVENTION

The invention had the object of finding novel compounds having valuable properties, in particular those which can be used for the preparation of medicaments.

The present invention relates to compounds in which the inhibition, regulation and/or modulation of kinase signal transduction, in particular tyrosine kinase signal transduction, plays a role, furthermore to pharmaceutical compositions which comprise these compounds, and to the use of the compounds for the treatment of kinase-induced diseases.

Specifically, the present invention relates to compounds which inhibit, regulate and/or modulate tyrosine kinase signal transduction, to compositions which comprise these compounds, and to methods for the use thereof for the treatment of tyrosine kinase-induced diseases and conditions, such as angiogenesis, cancer, tumour growth, arteriosclerosis, age-related macular degeneration, diabetic retinopathy, inflammatory diseases and the like, in mammals.

Tyrosine kinases are a class of enzymes which catalyse the transfer of the terminal phosphate of adenosine triphosphate to tyrosine residues in protein substrates. It is thought that tyrosine kinases, through substrate phosphorylation, play a crucial role in signal transduction for a number of cellular functions. Although the precise mechanisms of signal transduction are still unclear, tyrosine kinases have been shown to be important factors in cell proliferation, carcinogenesis and cell differentiation.

Tyrosine kinases can be categorised as receptor-type tyrosine kinases or non-receptor-type tyrosine kinases. Receptor-type tyrosine kinases have an extracellular portion, a transmembrane portion and an intracellular portion, while non-receptor-type tyrosine kinases are exclusively intracellular. Receptor-type tyrosine kinases consist of a multiplicity of transmembrane receptors with different biological activity. Thus, about 20 different subfamilies of receptor-type tyrosine kinases have been identified. One tyrosine kinase subfamily, known as the HER subfamily, consists of EGFR, HER2, HER3 and HER4. Ligands from this subfamily of receptors include epithelial growth factor, TGF-α, amphiregulin, HB-EGF, betacellulin and heregulin. Another subfamily of these receptor-type tyrosine kinases is the insulin subfamily, which includes INS-R, IGF-IR and IR-R. The PDGF subfamily includes the PDGF-α and -β receptor, CSFIR, c-kit and FLK-II. In addition, there is the FLK family, which consists of the kinase insert domain receptor (KDR), foetal liver kinase-1 (FLK-1), foetal liver kinase-4 (FLK-4) and fms tyrosine kinase-1 (fit-1). The PDGF and FLK family are usually discussed together due to the similarities between the two groups. For a detailed discussion of receptor-type tyrosine kinases, see the paoer by Plowman et al., DN & P 7(6):334-339, 1994, which is incorporated herein by way of reference.

Non-receptor-type tyrosine kinases likewise consist of a multiplicity of subfamilies, including Src, Frk, Btk, Csk, Abl, Zap70, Fes/Fps, Fak, Jak, Ack. and LIMK. Each of these subfamilies is further sub-divided into different receptors. For example, the Src subfamily is one of the largest subfamilies. It includes Src, Yes, Fyn, Lyn, Lck, Blk, Hck, Fgr and Yrk. The Src subfamily of enzymes has been linked to oncogenesis. For a more detailed discussion of non-receptor-type tyrosine kinases, see the paper by Bolen Oncogene, 8:2025-2031 (1993), which is hereby incorporated by way of reference.

Both receptor-type tyrosine kinases and non-receptor-type tyrosine kinases are involved in cellular signalling pathways leading to various conditions, including cancer, psoriasis and hyperimmune responses. It has been proposed that various receptor-type tyrosine kinases, and the growth factors binding to them, play a role in angiogenesis, although some may promote angiogenesis indirectly (Mustonen and Alitalo, J. Cell Biol. 129:895-898, 1995). One of these receptor-type tyrosine kinases is foetal liver kinase 1, also referred to as FLK-1. The human analogue of FLK-1 is the kinase insert domain-containing receptor KDR, which is also known as vascular endothelial cell growth factor receptor 2 or VEGFR-2, since it binds VEGF with high affinity. Finally, the murine version of this receptor has also been called NYK (Oelrichs et al., Oncogene 8(1):11-15, 1993). VEGF and KDR are a ligand-receptor pair which plays a vital role in the proliferation of vascular endothelial cells and the formation and sprouting of blood vessels, referred to as vasculogenesis and angiogenesis respectively.

Angiogenesis is characterised by excessive activity of vascular endothelial growth factor (VEGF). VEGF actually consists of a family of ligands (Klagsburn and D'Amore, Cytokine & Growth Factor Reviews 7:259-270, 1996). VEGF binds the high affinity membrane-spanning tyrosine kinase receptor KDR and the related fms tyrosine kinase-1, also known as Flt-1 or vascular endothelial cell growth factor receptor 1 (VEGFR-1). Cell culture and gene knockout experiments indicate that each receptor contributes to different aspects of angiogenesis. KDR mediates the mitogenic function of VEGF, whereas Flt-1 appears to modulate non-mitogenic functions, such as those associated with cellular adhesion. Inhibiting KDR thus modulates the level of mitogenic VEGF activity. In fact, tumour growth has been shown to be influenced by the antiangiogenic effect of VEGF receptor antagonists (Kim et al., Nature 362, pp. 841-844, 1993).

Solid tumours can therefore be treated with tyrosine inhibitors since these tumours depend on angiogenesis for the formation of the blood vessels that are necessary to support their growth. These solid tumours include monocytic leukaemia, carcinoma of the brain, urogenital tract, lymphatic system, stomach, larynx and lung, including lung adenocarcinoma and small cell lung carcinoma. Further examples include carcinomas in which overexpression or activation of Raf-activating oncogenes (for example K-ras, erb-B) is observed. These carcinomas include pancreatic and breast carcinoma. Inhibitors of these tyrosine kinases are therefore suitable for the prevention and treatment of proliferative diseases caused by these enzymes.

The angiogenic activity of VEGF is not limited to tumours. VEGF accounts for the angiogenic activity produced in or near the retina in diabetic retinopathy. This vascular growth in the retina leads to visual degeneration culminating in blindness. Ocular VEGF mRNA and protein levels are elevated by conditions such as retinal vein occlusion in primates and decreased pO₂ levels in mice that lead to neovascularisation. Intraocular injections of anti-VEGF monoclonal antibodies or VEGF receptor immunofusions inhibit ocular neovascularisation in both primate and rodent models. Irrespective of the cause of induction of VEGF in human diabetic retinopathy, inhibition of ocular VEGF is suitable for treating this disease.

Expression of VEGF is also significantly increased in hypoxic regions of animal and human tumours adjacent to areas of necrosis. In addition, VEGF is upregulated by the expression of the oncogenes ras, raf, src and p53 mutants (all of which are of importance in combating cancer). Anti-VEGF monoclonal antibodies inhibit the growth of human tumours in nude mice. Although the same tumour cells continue to express VEGF in culture, the antibodies do not diminish their mitotic rate. Thus, tumour-derived VEGF does not function as an autocrine mitogenic factor. VEGF therefore contributes to tumour growth in vivo by promoting angiogenesis through its paracrine vascular endothelial cell chemotactic and mitogenic activities. These monoclonal antibodies also inhibit the growth of typically less well vascularised human colon carcinomas in athymic mice and decrease the number of tumours arising from inoculated cells.

The expression of a VEGF-binding construct of Flk-1, Flt-1, the mouse KDR receptor homologue truncated to eliminate the cytoplasmic tyrosine kinase domains but retaining a membrane anchor, in viruses virtually stops the growth of a transplantable glioblastoma in mice, presumably by the dominant negative mechanism of heterodimer formation with membrane-spanning endothelial cell VEGF receptors. Embryonic stem cells, which normally grow as solid tumours in nude mice, do not produce detectable tumours if both VEGF alleles are knocked out. Taken together, these data indicate the role of VEGF in the growth of solid tumours. Inhibition of of KDR or Flt-1 is involved in pathological angiogenesis, and these receptors are suitable for the treatment of diseases in which angiogenesis is part of the overall pathology, for example inflammation, diabetic retinal vascularisation, as well as various forms of cancer, since tumour growth is known to be dependent on angiogenesis (Weidner et al., N. Engl. J. Med., 324, pp. 1-8, 1991).

Angiopoietin 1 (Ang1), a ligand for the endothelium-specific receptor-type tyrosine kinase TIE-2, is a novel angiogenic factor (Davis et al, Cell, 1996, 87:1161-1169; Partanen et al, Mol. Cell Biol., 12:1698-1707 (1992); U.S. Pat. Nos. 5,521,073; 5,879,672; 5,877,020; and 6,030,831). The acronym TIE stands for “tyrosine kinase with Ig and EGF homology domains”. TIE is used for the identification of a class of receptor-type tyrosine kinases which are expressed exclusively in vascular endothelial cells and early haemopoietic cells. TIE receptor kinases are typically characterised by the presence of an EGF-like domain and an immunoglobulin (IG)-like domain which consists of extracellular fold units stabilised by disulfide bridge bonds between the chains (Partanen et al Curr. Topics Microbiol. Immunol., 1999, 237:159-172). In contrast to VEGF, which exerts its function during the early stages of vascular development, Ang1 and its receptor TIE-2 act during the later stages of vascular development, i.e. during vascular transformation (transformation relates to the formation of a vascular lumen) and maturing (Yancopoulos et al, Cell, 1998, 93:661-664; Peters, K. G., Circ. Res., 1998, 83(3):342-3; Suri et al, Cell 87, 1171-1180 (1996)).

Accordingly, it would be expected that inhibition of TIE-2 should interrupt the transformation and maturing of a new vascular system initiated by angiogenesis and should thus interrupt the angiogenesis process. Furthermore, inhibition at the kinase domain binding site of VEGFR-2 would block phosphorylation of tyrosine residues and serve to interrupt initiation of angiogenesis. It must therefore be assumed that inhibition of TIE-2 and/or VEGFR-2 should prevent tumour angiogenesis and serve to slow or completely eliminate tumour growth.

Accordingly, treatment of cancer and other diseases associated with inappropriate angiogenesis could be provided.

The present invention is directed to methods for the regulation, modulation or inhibition of TIE-2 for the prevention and/or treatment of diseases in connection with unregulated or disturbed TIE-2 activity. In particular, the compounds according to the invention can also be employed in the treatment of certain forms of cancer. The compounds according to the invention can furthermore be used in order to provide additive or synergistic effects in certain existing cancer chemotherapies and/or can be used to restore the efficacy of certain existing cancer chemotherapies and irradiations.

Furthermore, compounds according to the invention can be used for the isolation and investigation of the activity or expression of TIE-2. In addition, they are particularly suitable for use in diagnostic methods for diseases in connection with unregulated or disturbed TIE-2 activity.

One of the principal mechanisms by which cellular regulation is effected is through the transduction of extracellular signals across the membrane that in turn modulate biochemical pathways within the cell. Protein phosphorylation represents one course by which intracellular signals are propagated from molecule to molecule, ultimately resulting in a cellular response. These signal transduction cascades are highly regulated and often overlap, as is evident from the existence of many protein kinases as well as phosphatases. Phosphorylation of proteins occurs predominantly at serine, threonine or tyrosine residues, and protein kinases have therefore been classified by their specificity of phosphorylation site, i.e. serine/threonine kinases and tyrosine kinases. Since phosphorylation is such a ubiquitous process within cells and since cellular phenotypes are largely influenced by the activity of these pathways, it is currently believed that a number of disease states and/or diseases are attributable to either aberrant activation or functional mutations in the molecular components of kinase cascades. Consequently, considerable attention has been devoted to the characterisation of these proteins and compounds that are able to modulate their activity (review see: Weinstein-Oppenheimer et al. Pharma. &. Therap., 2000, 88, 229-279 or Dancey and Sausville Nature Drug Discovery, 2003, 2, 296-313).

The identification of small compounds which specifically inhibit, regulate and/or modulate signal transduction of tyrosine kinases is therefore desirable and an aim of the present invention.

It has been found that the compounds according to the invention and salts thereof have very valuable pharmacological properties while being well tolerated.

In particular, they exhibit inhibiting properties in the case of tyrosine kinases.

As discussed herein, these signalling pathways are relevant for various diseases. Accordingly, the compounds according to the invention are suitable for the prophylaxis and/or treatment of diseases that are dependent on the said signalling pathways by interacting with one or more of the said signalling pathways.

The present invention therefore relates to compounds according to the invention as promoters or inhibitors, preferably as inhibitors, of the signalling pathways described herein. The invention therefore preferably relates to compounds according to the invention as promoters or inhibitors, preferably as inhibitors, of tyrosine kinase-dependent signal transmission pathways. The invention therefore preferably relates to compounds according to the invention as promoters or inhibitors, preferably as inhibitors, of TIE-2, VEGFR, PDGFR, FGFR and/or FLT/KDR.

In this connection, psoriasis, arthritis, inflammation, endometriosis, scarring, benign prostatic hyperplasia, immunological diseases, autoimmune diseases and immunodeficiency diseases are regarded as non-cancerous diseases, of which arthritis, inflammation, immunological diseases, autoimmune diseases and immunodeficiency diseases are usually regarded as non-hyperproliferative diseases. In this connection, brain cancer, lung cancer, squamous cell cancer, bladder cancer, gastric cancer, pancreatic cancer, hepatic cancer, renal cancer, colorectal cancer, breast cancer, head cancer, neck cancer, oesophageal cancer, gynaecological cancer, thyroid cancer, lymphomas, chronic leukaemia and acute leukaemia are to be regarded as cancerous diseases, all of which are usually regarded as hyperproliferative diseases. Especially cancerous cell growth and especially cancerous cell growth mediated by Raf kinase is a disease which is a target of the present invention. The present invention therefore relates to compounds according to the invention as medicaments and/or medicament active ingredients in the treatment and/or prophylaxis of the said diseases and to the use of compounds according to the invention for the preparation of a pharmaceutical for the treatment and/or prophylaxis of the said diseases as well as to a method for the treatment of the said diseases comprising the administration of one or more compounds according to the invention to a patient in need of such an administration.

It can be shown that the compounds according to the invention have an antiproliferative action in vivo in a xenotransplant tumour model. The compounds according to the invention are administered to a patient having a hyperproliferative disease, for example to inhibit tumour growth, to reduce inflammation associated with a lymphoproliferative disease, to inhibit transplant rejection or neurological damage due to tissue repair, etc. The present compounds are suitable for prophylactic or therapeutic purposes. As used herein, the term “treatment” is used to refer to both prevention of diseases and treatment of pre-existing conditions. The prevention of proliferation is achieved by administration of the compounds according to the invention prior to the development of overt disease, for example to prevent tumour growth, prevent metastatic growth, diminish restenosis associated with cardiovascular surgery, etc. Alternatively, the compounds are used for the treatment of ongoing diseases by stabilising or improving the clinical symptoms of the patient.

The host or patient can belong to any mammalian species, for example a primate species, particularly humans; rodents, including mice, rats and hamsters; rabbits; horses, cows, dogs, cats, etc. Animal models are of interest for experimental investigations, providing a model for the treatment of a human disease.

The susceptibility of a particular cell to treatment with the compounds according to the invention can be determined by in-vitro tests. Typically, a culture of the cell is combined with a compound according to the invention at various concentrations for a period of time which is sufficient to allow the active agents to induce cell death or to inhibit migration, usually between about one hour and one week. In-vitro testing can be carried out using cultivated cells from a biopsy sample. The viable cells remaining after the treatment are then counted.

The dose varies depending on the specific compound used, the specific disease, the patient status, etc. A therapeutic dose is typically sufficient considerably to reduce the undesired cell population in the target tissue while the viability of the patient is maintained. The treatment is generally continued until a considerable reduction has occurred, for example an at least about 50% reduction in the cell burden, and may be continued until essentially no more undesired cells are detected in the body.

For the identification of kinase inhibitors, various assay systems are available. In scintillation proximity assay (Sorg et al., J. of. Biomolecular Screening, 2002, 7, 11-19) and flashplate assay, the radioactive phosphorylation of a protein or peptide as substrate with γATP is measured. In the presence of an inhibitory compound, a decreased radioactive signal, or none at all, is detectable. Furthermore, homogeneous time-resolved fluorescence resonance energy transfer (HTR-FRET) and fluorescence polarisation (FP) technologies are suitable as assay methods (Sills et al., J. of Biomolecular Screening, 2002, 191-214).

Other non-radioactive ELISA assay methods use specific phospho-antibodies (phospho-ABs). The phospho-AB binds only the phosphorylated substrate. This binding can be detected by chemiluminescence using a second peroxidase-conjugated anti-sheep antibody (Ross et al., 2002, Biochem. J., just about to be published, manuscript BJ20020786).

There are many diseases associated with deregulation of cellular proliferation and cell death (apoptosis). The conditions of interest include, but are not limited to, the following. The compounds according to the invention are suitable for the treatment of a number of different conditions involving proliferation and/or migration of smooth muscle cells and/or inflammatory cells into the intimal layer of a vessel, resulting in restricted blood flow through that vessel, for example in the case of neointimal occlusive lesions. Occlusive graft vascular diseases of interest include atherosclerosis, coronary vascular disease after grafting, vein graft stenosis, peri-anastomatic prosthetic restenosis, restenosis after angioplasty or stent placement, and the like.

The compounds according to the invention are also suitable as p38 kinase inhibitors.

Other heteroarylureas which inhibit p38 kinase are described in WO 02/85859.

### PRIOR ART

WO 02/44156 describes benzimidazole derivatives other than TIE-2 and/or VEGFR2 inhibitors.

### SUMMARY OF THE INVENTION

The invention relates to compounds of the formula I 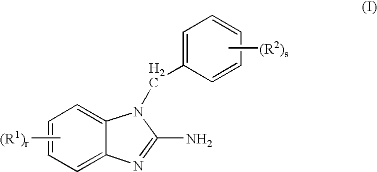
in which
R¹, R² can each, independently of one another, denote R, Hal, CN, NO₂, NHR, NR₂, NHCOR, NHSO₂R, OR, COR, CONHR, SCF₃, SO₃R, SO₂R, SO₂NR₂, SR, COOH or COOA, where two radicals R² together may also be —O—CH₂—O— or —O—CH₂—CH₂—O—,
R can denote H, A, Ar, Het, (CH₂)ₚAr, or (CH₂)ₚHet,
p can denote 1, 2 or 3
Ar can denote phenyl or naphthyl, each of which is unsubstituted or mono-, di- or trisubstituted by A, Hal, OH, OA, CN, NO₂, NH₂, NHA, NA₂, NHCOA, SCF₃, SO₂A, COOH, COOA, CONH₂, CONHA, CONA₂, NHSO₂A, SO₂NH₂, SO₂NHA, SO₂NA₂, CHO or COA,
A can denote unbranched, branched or cyclic alkyl having 1-10 C atoms, in which one or two CH₂ groups may be replaced by O or S atoms and/or by —CH═CH— groups and/or in addition 1-7 H atoms may be replaced by F and/or Cl and where A may be mono-, di- or trisubstituted by COOH, OH, COOA′ or CONH₂,
Het can denote a mono- or bicyclic saturated, unsaturated or aromatic heterocycle having 1 to 4 N, O and/or S atoms, which is unsubstituted, or may be mono-, di- or trisubstituted by carbonyl oxygen, Hal, A, —(CH₂)ₙ—Ar, —(CH₂)ₙ-cycloalkyl, OH, OA, NH₂, NHA, NA₂, NO₂, CN, COOH, COOA, CONH₂, CONHA, CONA₂, NHCOA, NHCONH₂, NHSO₂A, CHO, COA, SO₂NH₂ and/or S(O)ₘA,
A′ can be an unbranched, branched or cyclic alkyl having 1-6 C atoms,
m can denote 0, 1 or 2,
n can denote 0, 1, 2, 3 or 4,
Hal can denote F, Cl, Br, or I,
r can denote 0, 1, 2, 3 or 4,
s can denote 0, 1, 2, 3, 4 or 5,
   and pharmaceutically usable derivatives, salts, solvates and stereoisomers thereof, including mixtures thereof in all ratios.

The invention also relates to the optically active forms (stereoisomers), the enantiomers, the racemates, the diastereomers and the hydrates and solvates of these compounds. The term solvates of the compounds is taken to mean adductions of inert solvent molecules onto the compounds which form owing to their mutual attractive force. Solvates are, for example, mono- or dihydrates or alcoholates.

The term pharmaceutically usable derivatives is taken to mean, for example, the salts of the compounds according to the invention and also so-called prodrug compounds.

The term prodrug derivatives is taken to mean compounds of the formula I which have been modified by means of, for example, alkyl or acyl groups, sugars or oligopeptides and which are rapidly cleaved in the organism to form the effective compounds according to the invention.

These also include biodegradable polymer derivatives of the compounds according to the invention, as described, for example, in Int. J. Pharm. 115, 61-67 (1995).

The expression “effective amount” denotes the amount of a medicament or of a pharmaceutical active ingredient which causes in a tissue, system, animal or human a biological or medical response which is sought or desired, for example, by a researcher or physician.

In addition, the expression “therapeutically effective amount” denotes an amount which, compared with a corresponding subject who has not received this amount, results in the following:

improved treatment, healing, prevention or elimination of a disease, syndrome, disease condition, complaint, disorder or or side-effects or also the reduction in the progress of a disease, complaint or disorder.

The expression “therapeutically effective amount” also encompasses the amounts which are effective for increasing normal physiological function.

The invention also relates to mixtures of the compounds of the formula I according to the invention, for example mixtures of two diastereomers, for example in the ratio 1:1, 1:2, 1:3, 1:4, 1:5, 1:10, 1:100 or 1:1000. These are particularly preferably mixtures of stereoisomeric compounds.

For all radicals which occur more than once, such as, for example, R¹, their meanings are independent of one another.

Above and below, the radicals or parameters R¹, R² m and n have the meanings indicated for the formula 1, unless expressly stated otherwise.

Alkyl is unbranched (linear) or branched or cyclic, and has 1, 2, 3, 4, 5, 6, 7, 8, 9 or 10 C atoms.

A preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl. A very particularly preferably denotes alkyl having 1, 2, 3, 4, 5 or 6 C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl.

If A is cyclic, it preferably denotes cycloalkyl.

Cycloalkyl denotes, for example, cyclopropyl, cyclobutyl, cylopentyl, cyclohexyl or cycloheptyl.

A′ preferably denotes methyl, ethyl, propyl, isopropyl, butyl, isobutyl, sec-butyl, tert-butyl, pentyl, hexyl, trifluoromethyl, pentafluoroethyl or 1,1,1-trifluoroethyl.

R¹ preferably denotes R, COOH or COOA, where R is preferably H, A, Ar, Het, (CH₂)ₚAr, or (CH₂)ₚHet.

R² preferably denotes R, OR, NH₂, Hal, SO₂A or NHSO₂R, where two radicals R² together may also be —O—CH₂—O— or —O—CH₂—CH₂—O— and R is preferably H, A, Ar, Het, (CH₂)ₚAr, or (CH₂)ₚHet.

Het preferably denotes a mono- or bicyclic saturated, unsaturated or aromatic heterocycle having 1 to 4 N, O and/or S atoms, which is unsubstituted, or is mono-, di- or trisubstituted by carbonyl oxygen, Hal, A, —(CH₂)ₙ—Ar, —(CH₂)ₙ-cycloalkyl, OH, OA, NH₂, NHA, NA₂, NO₂, CN, COOH, COOA, CONH₂, CONHA, CONA₂, NHCOA, NHCONH₂, NHSO₂A, CHO, COA, SO₂NH₂ and/or S(O)ₘA.

r preferably denotes 1, 2 or 3.

r preferably denotes 0 or 1.

Ar denotes, for example, phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-acetamidophenyl, o-, m- or p-methoxy-phenyl, o-, m- or p-ethoxyphenyl, o-, m- or p-ethoxycarbonylphenyl, o-, m- or p-(N,N-dimethylamino)phenyl, o-, m- or p-(N,N-dimethylaminocarbonyl)phenyl, o-, m- or p-(N-ethylamino)phenyl, o-, m- or p-(N,N-diethylamino)phenyl, o-, m- or p-fluorophenyl, o-, m- or p-bromophenyl, o-, m- or p-chlorophenyl, o-, m- or p-(methylsulfonamido)phenyl, o-, m- or p-(methylsulfonyl)phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl, furthermore, for example, 4-phenylphenyl.

Ar preferably denotes, for example, phenyl which is unsubstituted or mono- or disubstituted by Hal, A, OA, SO₂A, COOR², SO₂NH₂ or CN.

Ar very particularly preferably denotes phenyl which is unsubstituted or mono- or disubstituted by A and/or Hal.

Unsubstituted Het denotes, for example, 2- or 3-furyl, 2- or 3-thienyl, 1-, 2- or 3-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7- benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazoliinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxan-6-yl, 2,1,3-benzothiadiazol-4- or -5-yl or 2,1 ,3-benzoxadiazol-5-yl.

The heterocyclic radicals may also be partially or fully hydrogenated. Het can thus also denote, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl or 2,3-dihydro-2-oxofuranyl.

The formula I preferably has the following formulae Ia-II

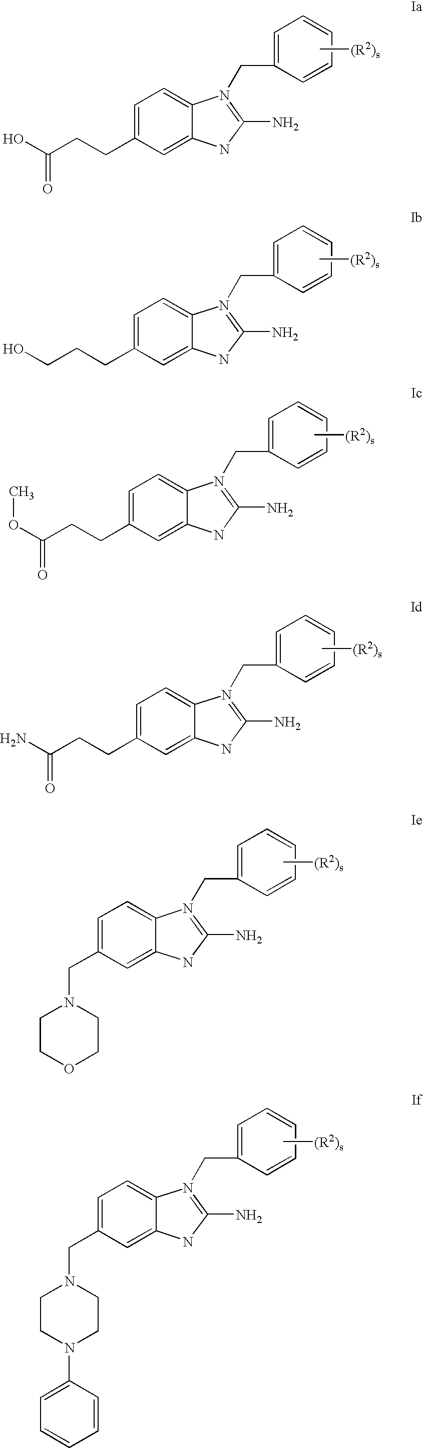

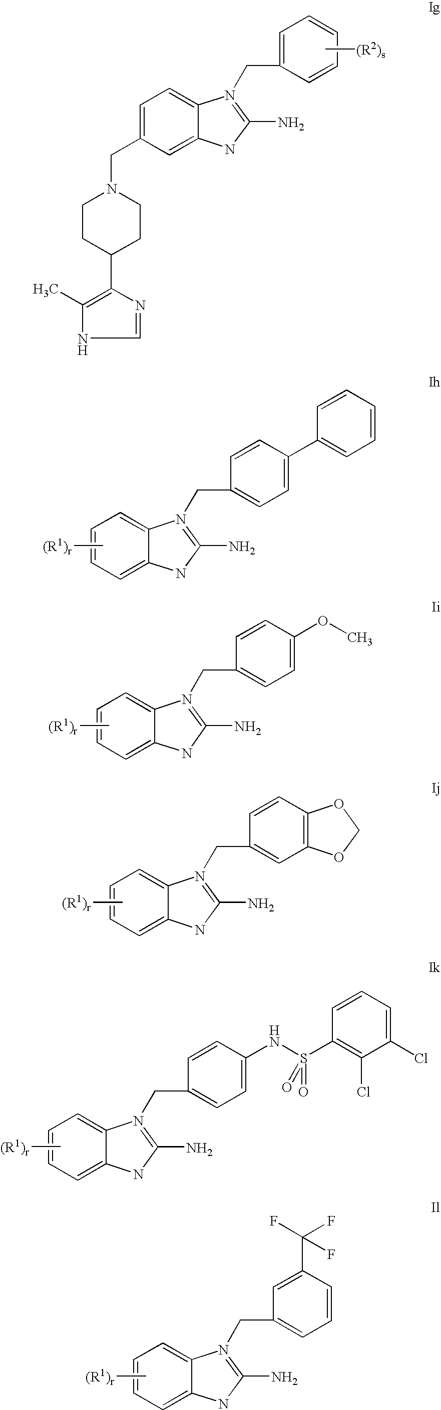

Some preferred groups of compounds can be expressed by the following sub-formulae Iaa to Iag, which conform to the formula I and in which the radicals not designated in greater detail have the meaning indicated in the case of the formula I, but in which
in Iaa
R¹ denotes R, COOH or COOA;
in Iab
R² denotes R, OR, NH₂, Hal, SO₂A or NHSO₂R, where two radicals R² together may also be —O—CH₂—O— or —O—CH₂—CH₂—O—;
in Iac Ar denotes phenyl which is unsubstituted or mono-, di- or trisubstituted by Hal;
in Iad A denotes unbranched, branched or cyclic alkyl having 1, 2, 3, 4, 5 or 6 C atoms, in which, in addition, 1-7 H atoms may be replaced by F and/or Cl, where A may also be mono-, di- or trisubstituted by COOH, OH, COOA′ or CONH₂;
in Iae
R¹ denote R, COOH or COOA;
R² denotes R, OR, NH₂, Hal, SO₂A or NHSO₂R, where two radicals R² together may also be —O—CH₂—O— or —O—CH₂—CH₂—O—,
R denotes H, A, Ar, Het, (CH₂)ₚAr, or (CH₂)ₚHet,
p denotes 1, 2 or 3,
Ar denotes phenyl or naphthyl, each of which is unsubstituted or mono-, di- or trisubstituted by A, Hal, OH, OA, CN, NO₂, NH₂, NHA, NA₂, NHCOA, SCF₃, SO₂A, COOH, COOA, CONH₂, CONHA, CONA₂, NHSO₂A, SO₂NH₂, SO₂NHA, SO₂NA₂, CHO, COA,
A denotes unbranched, branched or cyclic alkyl having 1-10 C atoms, in which one or two CH₂ groups may be replaced by O or S atoms and/or by —CH═CH— groups and/or in addition 1-7 H atoms may be replaced by F and/or Cl and where A may be mono-, di- or trisubstituted by COOH, OH, COOA′ or CONH₂,
A′ denotes unbranched, branched or cyclic alkyl having 1-6 C atoms,
Hal denotes F, Cl, Br, or I,
r denotes 0, 1, 2, 3 or 4,
s denotes 0, 1, 2, 3, 4 or 5;
in Iaf
R¹ denote A, (CH₂)ₚHet, COOH, or COOA,
Het denote a mono- or bicyclic saturated, unsaturated or aromatic heterocycle having 1 to 4 N, O and/or S atoms, which is unsubstituted, or may be mono-, di- or trisubstituted by carbonyl oxygen, Hal, A, —(CH₂)ₙ—Ar, —(CH₂)ₙ-cycloalkyl, OH, OA, NH₂, NHA, NA₂, NO₂, CN, COOH, COOA, CONH₂, CONHA, CONA₂, NHCOA, NHCONH₂, NHSO₂A, CHO, COA, SO₂NH₂ and/or S(O)ₘA and
in Iag
R² can be Ar, OA, Hal, A, NHSO₂Ar, NH₂, SO₂A, where two radicals R² together may also be —O—CH₂—O— or —O—CH₂—CH₂—O—,
   and pharmaceutically usable derivatives, solvates and stereoisomers thereof, including mixtures thereof in all ratios.

The compounds according to the invention and also the starting materials for the preparation thereof are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ so that they are not isolated from the reaction mixture, but instead are immediately converted further into the compounds according to the invention.

The starting compounds are generally known. If they are novel, however, they can be prepared by methods known per se.

Compounds of the formula I can preferably be obtained by reacting aniline derivatives of the formula II (J. Med Chem. 1992, 35, page 877-885, THL 2000, 41, page 9871-9874) with cyanogen bromide.

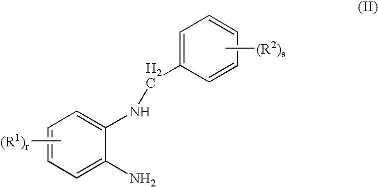

The reaction is carried out by methods which are known to the person skilled in the art.

Firstly, reaction takes place in a suitable solvent, if desired in the presence of an organic base, such as, for example, triethylamine, or an inorganic base, such as, for example, an alkali or alkaline earth metal carbonate.

Suitable inert solvents are, for example, hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether (methyl glycol or ethyl glycol), ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Depending on the conditions used, the reaction time is between a few minutes and 14 days, the reaction temperature is between about −30° and 140°, normally between −10° and 90°, in particular between about 0° and about 70°.

A base of the compounds of the formula I according to the invention can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, sulfamic acid, furthermore organic acids, in particular aliphatic, alicyclic, araliphatic, aromatic or heterocyclic mono- or polybasic carboxylic, sulfonic or sulfuric acids, for example formic acid, acetic acid, trifluoroacetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, laurylsulfuric acid. Salts with physiologically unacceptable acids, for example picrates, can be used for the isolation and/or purification of the compounds according to the invention.

On the other hand, compounds of the formula I can be converted using bases (for example sodium hydroxide or carbonate or potassium hydroxide or carbonate) into the corresponding metal salts, in particular alkali metal or alkaline earth metal salts, or into the corresponding ammonium salts.

Physiologically acceptable organic bases, such as, for example, ethanolamine, can also be used.

The invention furthermore relates to the use of the compounds and/or physiologically acceptable salts thereof for the preparation of a medicament (pharmaceutical composition), in particular by non-chemical methods. They can be converted into a suitable dosage form here together with at least one solid, liquid and/or semi-liquid excipient or adjuvant and, if desired, in combination with one or more further active ingredients.

The invention furthermore relates to medicaments comprising at least one compound according to the invention and/or pharmaceutically usable derivatives, solvates and stereoisomers thereof, including mixtures thereof in all ratios, and optionally excipients and/or adjuvants.

Pharmaceutical formulations can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials, and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a prespecified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds according to the invention and salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds according to the invention and the salts, solvates and physiologically functional derivatives thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, where the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insuffiators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in the freeze-dried (lyophilised) state, so that only the addition of the sterile carrier liquid, for example water for injection purposes, immediately before use is necessary.

Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the present invention depends on a number of factors, including, for example, the age and weight of the animal, the precise disease condition which requires treatment, and its severity, the nature of the formulation and the method of administration, and is ultimately determined by the treating doctor or vet. However, an effective amount of a compound according to the invention for the treatment of neoplastic growth, for example colon or breast carcinoma, is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of the other conditions mentioned above.

The invention furthermore relates to medicaments comprising at least one compound according to the invention and/or pharmaceutically usable derivatives, solvates and stereoisomers thereof, including mixtures thereof in all ratios, and at least one further medicament active ingredient.

The invention also relates to a set (kit) consisting of separate packs of
(a) an effective amount of a compound according to the invention and/or pharmaceutically usable derivatives, solvates and stereoisomers thereof, including mixtures thereof in all ratios, and
(b) an effective amount of a further medicament active ingredient.

The set comprises suitable containers, such as boxes, individual bottles, bags or ampoules. The set may, for example, comprise separate ampoules, each containing an effective amount of a compound according to the invention and/or pharmaceutically usable derivatives, solvates and stereoisomers thereof, including mixtures thereof in all ratios, and an effective amount of a further medicament active ingredient in dissolved or lyophilised form.

Use

The present compounds are suitable as pharmaceutical active ingredients for mammals, especially for humans, in the treatment of tyrosine kinase-induced diseases. These diseases include the proliferation of tumour cells, pathological neovascularisation (or angiogenesis) which promotes the growth of solid tumours, ocular neovascularisation (diabetic retinopathy, age-related macular degeneration and the like) and inflammation (psoriasis, rheumatoid arthritis and the like).

The present invention encompasses the use of the compounds according to the invention according to Claim 1 and/or physiologically acceptable salts and solvates thereof for the preparation of a medicament for the treatment or prevention of cancer. Preferred carcinomas for the treatment originate from the group cerebral carcinoma, urogenital tract carcinoma, carcinoma of the lymphatic system, stomach carcinoma, laryngeal carcinoma and lung carcinoma. A further group of preferred forms of cancer are monocytic leukaemia, lung adenocarcinoma, small cell lung carcinomas, pancreatic cancer, glioblastomas and breast carcinoma.

Also encompassed is the use of the compounds according to the invention according to Claim 1 and/or physiologically acceptable salts and solvates thereof for the preparation of a medicament for the treatment or prevention of a disease in which angiogenesis is implicated.

Such a disease in which angiogenesis is implicated is an eye disease, such as retinal vascularisation, diabetic retinopathy, age-induced macular degeneration and the like.

The use of compounds according to the invention according to Claim 1 and/or physiologically acceptable salts and solvates thereof for the preparation of a medicament for the treatment or prevention of inflammatory diseases also falls within the scope of the present invention. Examples of such inflammatory diseases include rheumatoid arthritis, psoriasis, contact dermatitis, delayed hypersensitivity reaction and the like.

Also encompassed is the use of the compounds according to the invention according to Claim 1 and/or physiologically acceptable salts and solvates thereof for the preparation of a medicament for the treatment or prevention of a tyrosine kinase-induced disease or a tyrosine kinase-induced condition in a mammal, where this method a therapeutically effective amount of a compound according to the invention is administered to a sick mammal in need of such treatment. The therapeutic amount depends on the particular disease and can be determined by the person skilled in the art without undue effort.

The present invention also encompasses the use of the compounds according to the invention according to Claim 1 and/or physiologically acceptable salts and solvates thereof for the preparation of a medicament for the treatment or prevention of retinal vascularisation.

Methods for the treatment or prevention of eye diseases, such as diabetic retinopathy and age-related macular degeneration, are likewise part of the invention. The use for the treatment or prevention of inflammatory diseases, such as rheumatoid arthritis, psoriasis, contact dermatitis and delayed hypersensitivity reaction, as well as the treatment or prevention of bone pathologies from the group osteosarcoma, osteoarthritis and rickets, likewise falls within the scope of the present invention.

The expression “tyrosine kinase-induced diseases or conditions” refers to pathological conditions that depend on the activity of one or more tyrosine kinases. Tyrosine kinases participate either directly or indirectly in the signal transduction pathways of a variety of cellular activities, including proliferation, adhesion and migration and differentiation. Diseases associated with tyrosine kinase activity include proliferation of tumour cells, pathological neovascularisation that promotes the growth of solid tumours, ocular neovascularisation (diabetic retinopathy, age-related macular degeneration and the like) and inflammation (psoriasis, rheumatoid arthritis and the like).

The compounds according to the invention according to Claim 1 can be administered to patients for the treatment of cancer. The present compounds inhibit tumour angiogenesis, thereby affecting the growth of tumours (J. Rak et al. Cancer Research, 55:4575-4580, 1995). The angiogenesis-inhibiting properties of the present compounds according to Claim 1 are also suitable for the treatment of certain forms of blindness related to retinal neovascularisation.

The compounds according to Claim 1 are also suitable for the treatment of certain bone pathologies, such as osteosarcoma, osteoarthritis and rickets, also known as oncogenic osteomalacia (Hasegawa et al., Skeletal Radiol. 28, pp. 41-45, 1999; Gerber et al., Nature Medicine, Vol. 5, No. 6, pp. 623-628, June 1999). Since VEGF directly promotes osteoclastic bone resorption through KDR/Flk-1 expressed in mature osteoclasts (FEBS Let. 473:161-164 (2000); Endocrinology, 141:1667 (2000)), the present compounds are also suitable for the treatment and prevention of conditions related to bone resorption, such as osteoporosis and Paget's disease. The compounds can also be used for the reduction or prevention of tissue damage which occurs after cerebral ischaemic events, such as strokes, by reducing cerebral oedema, tissue damage and reperfusion injury following ischaemia (Drug News Perspect 11:265-270 (1998); J. Clin. Invest. 104:1613-1620 (1999)).

The invention thus relates to the use of compounds according to Claim 1, and pharmaceutically usable derivatives, solvates and stereoisomers thereof, including mixtures thereof in all ratios, for the preparation of a medicament for the treatment of diseases in which the inhibition, regulation and/or modulation of kinase signal transduction plays a role.

Preference is given here to kinases selected from the group of tyrosine kinases.

The tyrosine kinases are preferably TIE-2, VEGFR, PDGFR, FGFR and/or FLT/KDR.

Preference is given to the use of compounds according to Claim 1, and pharmaceutically usable derivatives, solvates and stereoisomers thereof, including mixtures thereof in all ratios,

for the preparation of a medicament for the treatment of diseases which are influenced by inhibition of tyrosine kinases by the compounds according to Claim 1.

Particular preference is given to the use for the preparation of a medicament for the treatment of diseases which are influenced by inhibition of TIE-2, VEGFR, PDGFR, FGFR and/or FLT/KDR by the compounds according to Claim 1.

Especial preference is given to the use for the treatment of a disease where the disease is a solid tumour.

The solid tumour is preferably selected from the group of tumours of the squamous epithelium, bladder, kidneys, head and neck, oesophagus, cervix, thyroid, intestine, liver, brain, prostate, urogenital tract, lymphatic system, stomach, larynx and/or lung.

The solid tumour is furthermore preferably selected from the group monocytic leukaemia, lung adenocarcinoma, small cell lung carcinomas, pancreatic cancer, glioblastomas and breast carcinoma.

The invention furthermore relates to the use of the compounds according to the invention for the treatment of a disease in which angiogenesis is involved.

The disease is preferably an eye disease.

The invention furthermore relates to the use for the treatment of retinal vascularisation, diabetic retinopathy, age-induced macular degeneration and/or inflammatory diseases.

The inflammatory disease is preferably selected from the group rheumatoid arthritis, psoriasis, contact dermatitis and delayed hypersensitivity reaction.

The invention furthermore relates to the use of the compounds according to the invention for the treatment of bone pathologies, where the bone pathology originates from the group osteosarcoma, osteoarthritis and rickets.

These are cancerous diseases or non-cancerous diseases.

The non-cancerous diseases are selected from the group consisting of psoriasis, arthritis, inflammation, endometriosis, scarring, benign prostatic hyperplasia, immunological diseases, autoimmune diseases and immunodeficiency diseases.

The cancerous diseases are selected from the group consisting of brain cancer, lung cancer, squamous cell cancer, bladder cancer, gastric cancer, pancreatic cancer, hepatic cancer, renal cancer, colorectal cancer, breast cancer, head cancer, neck cancer, oesophageal cancer, gynaecological cancer, thyroid cancer, lymphoma, chronic leukaemia and acute leukaemia.

The compounds according to the invention may also be administered at the same time as other well-known therapeutic agents that are selected for their particular usefulness against the condition that is being treated. For example, in the case of bone conditions, combinations that would be favourable include those with antiresorptive bisphosphonates, such as alendronate and risedronate, integrin blockers (as defined further below), such as αvβ3 antagonists, conjugated oestrogens used in hormone replacement therapy, such as Prempro®, Premarin® and Endometrion®; selective oestrogen receptor modulators (SERMs), such as raloxifene, droloxifene, CP-336, 156 (Pfizer) and lasofoxifene, cathepsin K inhibitors, and ATP proton pump inhibitors.

The present compounds are also suitable for combination with known anti-cancer agents. These known anti-cancer agents include the following: oestrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors and other angiogenesis inhibitors. The present compounds are particularly suitable for administration at the same time as radiotherapy. The synergistic effects of inhibiting VEGF in combination with radiotherapy have been described in the art (see WO 00/61186).

“Oestrogen receptor modulators” refers to compounds which interfere with or inhibit the binding of oestrogen to the receptor, regardless of mechanism. Examples of oestrogen receptor modulators include, but are not limited to, tamoxifen, raloxifene, idoxifene, LY353381, LY 117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]phenyl 2,2-dimethylpropanoate, 4,4′-dihydroxybenzophenone-2,4-dinitrophenylhydrazone and SH646.

“Androgen receptor modulators” refers to compounds which interfere with or inhibit the binding of androgens to the receptor, regardless of mechanism. Examples of androgen receptor modulators include finasteride and other 5a-reductase inhibitors, nilutamide, flutamide, bicalutamide, liarozole and abiraterone acetate.

“Retinoid receptor modulators” refers to compounds which interfere with or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl)retinamide and N-4-carboxyphenyl retinamide.

“Cytotoxic agents” refers to compounds which result in cell death primarily through direct action on the cellular function or inhibit or interfere with cell myosis, including alkylating agents, tumour necrosis factors, intercalators, microtubulin inhibitors and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPX100, (trans,trans,trans)bis-mu-(hexane-1,6-diamine)mu-[diamineplati-num(II)]bis[diamine(chloro)platinum(II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755 and 4-demethoxy-3-deamino-3-aziridinyl-4-methylsulfonyldaunorubicin (see WO 00/50032).

Examples of microtubulin inhibitors include paclitaxel, vindesine sulfate, 3′,4′-didehydro-4′-deoxy-8′-norvincaleu koblastine, docetaxol, rhizoxin, dolastatin, mivobulin isethionate, auristatin, cemadotin, RPR¹⁰⁹⁸⁸¹, BMS184476, vinflunine, cryptophycin, 2,3,4,5,6-pentafluoro-N-(3-fluoro-4-methoxyphenyl)benzenesulfonamide, anhydrovinblastine, N,N-dimethyl-L-valyl-L-valyl-N-methyl-L-valyl-L-prolyl-L-proline t-butylamide, TDX258 and BMS188797.

Some examples of topoisomerase inhibitors are topotecan, hycaptamine, irinotecan, rubitecan, 6-ethoxypropionyl-3′,4′-O-exobenzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]indolizino[1,2b]quinoline-10,13(9H,15H)dione, lurtotecan, 7-[2-(N-isopropylamino)ethyl]-(20S)-camptothecin, BNP1350, BNPI1100, BN80915, BN80942, etoposide phosphate, teniposide, sobuzoxane, 2′-dimethylamino-2′-deoxyetoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one and dimesna.

“Antiproliferative agents” include antisense RNA and DNA oligonucleotides, such as G3139, ODN698, RVASKRAS, GEM231 and INX3001, and antimetabolites, such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, neizarabine, 2′-deoxy-2′-methylidenecytidine, 2′-fluoromethylene-2′-deoxycytidine, N-[5-(2,3-dihydrobenzofuryl)sulfonyl]-N′-(3,4-dichlorophenyl)urea, N6-[4-deoxy-4-[N2-[2(E),4(E)-tetradecadienoyl]glycylamino]-L-glycero-B-L-mannohepto-pyranosyl]adenine, aplidine, ecteinascidin, troxacitabine, 4-[2-amino-4-oxo-4,6,7,8-tetrahydro-3H-pyrimidino[5,4-b]-1,4-thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)tetradeca-2,4,6-trien-9-ylacetic acid ester, swainsonine, lometrexol, dexrazoxane, methioninase, 2′-cyano-2′-deoxy-N4-palmitoyl-1-B-D-arabinofuranosyl cytosine and 3-aminopyridine-2-carboxaldehyde thiosemicarbazone. “Antiproliferative agents” also include monoclonal antibodies to growth factors other than those listed above under “angiogenesis inhibitors”, such as trastuzumab, and tumour suppressor genes, such as p53, which can be delivered via recombinant virus-mediated gene transfer (see U.S. Pat. No. 6,069,134, for example).

Assays

The compounds according to the invention described in the examples were tested by the assays described below and were found to have a kinase-inhibiting activity. Other assays are known from the literature and could readily be performed by the person skilled in the art (see, for example, Dhanabal et al., Cancer Res. 59:189-197; Xin et al., J. Biol. Chem. 274:9116-9121; Sheu et al., Anticancer Res. 18:4435-4441; Ausprunk et al., Dev. Biol. 38:237-248; Gimbrone et al., J. Natl. Cancer Inst. 52:413-427; Nicosia et al., In Vitro 18:538-549).

VEGF Receptor Kinase Assay

VEGF receptor kinase activity is measured by incorporation of radiolabelled phosphate into 4:1 polyglutamic acid/tyrosine substrate (pEY). The phosphorylated pEY product is trapped onto a filter membrane, and the incorporation of radiolabelled phosphate is quantified by scintillation counting.

Materials

VEGF Receptor Kinase

The intracellular tyrosine kinase domains of human KDR (Terman, B. I. et al. Oncogene (1991) Vol. 6, pp. 1677-1683.) and Flt-1 (Shibuya, M. et al. Oncogene (1990) Vol. 5, pp. 519-524) were cloned as glutathione S-transferase (GST) gene fusion proteins. This was accomplished by cloning the cytoplasmic domain of the KDR kinase as an in-frame fusion at the carboxyl terminus of the GST gene. Soluble recombinant GST-kinase domain fusion proteins were expressed in Spodoptera frugiperda (Sf21) insect cells (Invitrogen) using a baculovirus expression vector (pAcG2T, Pharmingen).

Lysis Buffer

50 mM Tris pH 7.4, 0.5 M NaCl, 5 mM DTT, 1 mM EDTA, 0.5% of Triton X-100, 10% of glycerol, 10 mg/ml each of leupeptin, pepstatin and aprotinin and 1 mM phenylmethylsulfonyl fluoride (all Sigma).

Wash Buffer

50 mM Tris pH 7.4, 0.5 M NaCl, 5 mM DTT, 1 mM EDTA, 0.05% of Triton X-100, 10% of glycerol, 10 mg/ml each of leupeptin, pepstatin and aprotinin and 1 mM phenylmethylsulfonyl fluoride.

Dialysis Buffer

50 mM Tris pH 7.4, 0.5 M NaCl, 5 mM DTT, 1 mM EDTA, 0.05% of Triton X-100, 50% of glycerol, 10 mg/ml each of leupeptin, pepstatin and aprotinin and 1 mM phenylmethylsulfonyl fluoride.

10× Reaction Buffer

200 mM Tris, pH 7.4, 1.0 M NaCl, 50 mM MnCl₂, 10 mM DTT and 5 mg/ml bovine serum albumin [BSA] (Sigma).

Enzyme Dilution Buffer

50 mM Tris, pH 7.4, 0.1 M NaCl, 1 mM DTT, 10% of glycerol, 100 mg/ml BSA.

10× Substrate

750 μg/ml poly(glutamic acid/tyrosine; 4:1) (Sigma).

Stop Solution

30% trichloroacetic acid, 0.2 M sodium pyrophosphate (both Fisher).

Wash Solution

15% trichloroacetic acid, 0.2 M sodium pyrophosphate.

Filter Plates

Millipore #MAFC NOB, GF/C glass-fibre 96-well plate.

Method A—Protein Purification

1. Sf21 cells were infected with recombinant virus at a multiplicity of infection of 5 virus particles/cell and grown at 27° C. for 48 hours.

2. All steps were performed at 4° C. Infected cells were harvested by centrifugation at 1000×g and lysed at 4° C. for 30 minutes with 1/10 volume of lysis buffer followed by centrifugation at 100.000×g for 1 hour. The supernatant was then passed over a glutathione Sepharose column (Pharmacia) equilibrated with lysis buffer and washed with 5 volumes of the same buffer followed by 5 volumes of wash buffer. Recombinant GST-KDR protein was eluted with wash buffer/10 mM reduced glutathione (Sigma) and dialysed against dialysis buffer.

Method B—VEGF Receptor Kinase Assay

1. Add 5 μl of inhibitor or control to the assay in 50% DMSO.

2. Add 35 μl of reaction mixture containing 5 μl of 10× reaction buffer, 5 μl of 25 mM ATP/10 μCi[³³P]ATP (Amersham) and 5 μl of 10× substrate.

3. Start reaction by addition of 10 μl of KDR (25 nM) in enzyme dilution buffer.

4. Mix and incubate at room temperature for 15 minutes.

5. Stop reaction by addition of 50 μl of stop solution.

6. Incubate at 4° C. for 15 minutes.

7. Transfer 90 μl aliquot to filter plate.

8. Aspirate and wash 3 times with wash solution.

9. Add 30 μl of scintillation cocktail, seal plate and count in a Wallac Microbeta scintillation counter.

Human Umbilical Vein Endothelial Cell Mitogenesis Assay

Expression of VEGF receptors that mediate mitogenic responses to the growth factor is largely restricted to vascular endothelial cells. Human umbilical vein endothelial cells (HUVECs) in culture proliferate in response to VEGF treatment and can be used as an assay system to quantify the effects of KDR kinase inhibitors on VEGF stimulation. In the assay described, quiescent HUVEC monolayers are treated with vehicle or test compound 2 hours prior to addition of VEGF or basic fibroblast growth factor (bFGF). The mitogenic response to VEGF or bFGF is determined by measuring the incorporation of [³H]thymidine into cellular DNA.

Materials

HUVECs

HUVECs frozen as primary culture isolates are obtained from Clonetics Corp. The cells are maintained in endothelial growth medium (EGM; Clonetics) and are used for mitogenic assays in passages 3-7.

Culture Plates

NUNCLON 96-well polystyrene tissue culture plates (NUNC #167008).

Assay Medium

Dulbecco's modification of Eagle's medium containing 1 g/ml glucose (low-glucose DMEM; Mediatech) plus 10% (v/v) foetal bovine serum (Clonetics).

Test Compounds

Working stock solutions of test compounds are diluted serially in 100% dimethyl sulfoxide (DMSO) to 400 times greater than their desired final concentration. The final dilutions (concentration 1×) are prepared with assay medium immediately prior to addition to the cells.

10× Growth Factors

Solutions of human VEGF 165 (500 ng/ml; R&D Systems) and bFGF (10 ng/ml; R&D Systems) are prepared with assay medium.

10×[³H]thymidine

[Methyl-³H]thymidine (20 Ci/mmol; Dupont-NEN) is diluted to 80 μCi/ml in low-glucose DMEM medium.

Cell Wash Medium

Hank's balanced salt solution (Mediatech) containing 1 mg/ml bovine serum albumin (Boehringer-Mannheim).

Cell Lysis Solution

1 N NaOH, 2% (w/v) of Na₂CO₃.

Method 1

HUVEC monolayers maintained in EGM are harvested by trypsinisation and plated out at a density of 4000 cells per 100 μl of assay medium per well in 96-well plates. Cell growth is arrested for 24 hours at 37° C. in a humidified atmosphere containing 5% of CO₂.

Method 2

Growth-arrest medium is replaced by 100 μl of assay medium containing either vehicle (0.25% [v/v] of DMSO) or the desired final concentration of test compound. All determinations are performed in triplicate. Cells are then incubated at 37° C./5% CO₂ for 2 hours to allow test compounds to enter cells.

Method 3

After pre-treatment for 2 hours, cells are stimulated by addition of 10 μl/well of either assay medium, 10×VEGF solution or 10×bFGF solution. Cells are then incubated at 37° C./5% CO₂.

Method 4

After 24 hours in the presence of growth factors, 10×[³H]thymidine (10 μl/well) is added.

Method 5

Three days after addition of [³H]thymidine, medium is removed by aspiration, and cells are washed twice with cell wash medium (400 μl/well followed by 200 μl/well). The washed, adherent cells are then solubilised by addition of cell lysis solution (100 μl/well) and warming to 37° C. for 30 minutes. Cell lysates are transferred into 7 ml glass scintillation vials containing 150 μl of water. Scintillation cocktail (5 ml/vial) is added, and cell-associated radioactivity is determined by liquid scintillation spectroscopy. According to these assays, the compounds of the formula I are inhibitors of VEGF and are thus suitable for the inhibition of angiogenesis, such as in the treatment of eye diseases, for example diabetic retinopathy, and for the treatment of carcinomas, for example solid tumours. The present compounds inhibit VEGF-stimulated mitogenesis of human vascular endothelial cells in culture with IC50 values of 0.01-5.0 μM. These compounds also show selectivity over related tyrosine kinases (for example FGFR1 and the Src family; for relationship between Src kinases and VEGFR kinases, see Eliceiri et al., Molecular Cell, Vol. 4, pp. 915-924, December 1999).

The TIE-2 tests can be carried out, for example, analogously to the methods indicated in WO 02/44156.

The assay determines the inhibiting activity of the substances to be tested in the phosphorylation of the substrate poly(Glu, Tyr) by Tie-2 kinase in the presence of radioactive ³³P-ATP. The phosphorylated substrate binds to the surface of a “flashplate” microtitre plate during the incubation time. After removal of the reaction mixture, the microtitre plate is washed a number of times and the radioactivity on its surface is subsequently measured. An inhibiting effect of the substances to be measured results in lower radioactivity compared with an undisturbed enzymatic reaction.

Above and below, all temperatures are indicated in ° C. In the following examples, “conventional work-up” means: water is added if necessary, the pH is adjusted, if necessary, to values between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rf values on silica gel; eluent: ethyl acetate/methanol 9:1.
Mass spectrometry (MS): EI (electron impact ionisation) M⁺ FAB (fast atom bombardment) (M+H)⁺ ESI (electrospray ionisation) (M+H)⁺ (unless stated otherwise)

### EXAMPLE 1

### Synthesis of 3-[2-amino-1-(4-methoxybenzyl)-1H-benzimidazol-5-yl]propionic acid (A) and 3-[2-amino-1-(4-methoxybenzyl)-1H-benzimidazol-5-yl]-propan-1-ol (B)

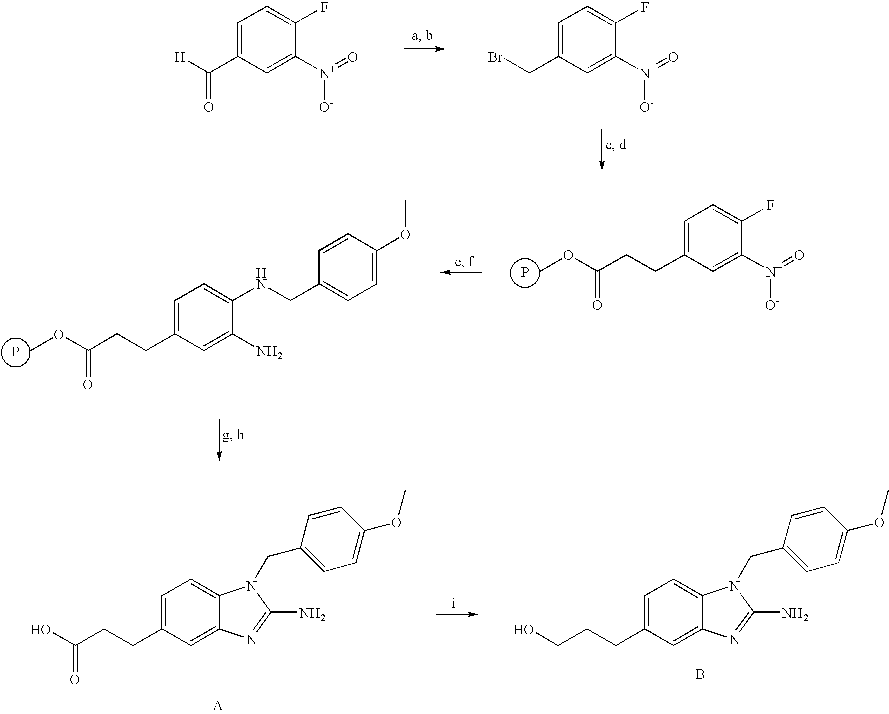

a

1.69 g (0.01 mol) of 4-fluoro-3-nitrobenzaldehyde are suspended in 100 ml of n-heptane under argon. 20 g of silica gel 60 (0.063-0.200 mm) and 0.378 g (0.01 mol) of sodium borohydride (fine granules, for synthesis) are then added, and the mixture is stirred at 40° C. for 90 minutes. The mixture is then filtered, and the organic phase is evaporated to dryness (under reduced pressure), leaving 1.7 g of 4-fluoro-3-nitrobenzyl alcohol.

b

1.3 g (7.6 mmol) of 4-fluoro-3-nitrobenzyl alcohol are dissolved in 20 ml of dichloromethane. 2.49 ml of bromotrimethylsilane for synthesis are added, and the mixture is stirred overnight at room temperature. The reaction mixture is evaporated to dryness under reduced pressure and purified via a silica-gel column (eluent PE/EA 4:1), giving 1.7 g of 4-fluoro-3-nitrobenzyl bromide.

c

4.2 g of sodium hydride (60% suspension in paraffin oil) for synthesis are initially introduced in 150 ml of THF under argon. A mixture of 16.02 ml of diethyl malonate in 50 ml of THF is added dropwise, and the mixture is stirred at room temperature for 5 minutes. 5.0 g of 4-fluoro-3-nitrobenzyl bromide are then dissolved in 50 ml of THF and likewise added dropwise. The mixture is stirred at room temperature for 15 minutes. The reaction mixture is diluted with 200 ml of both dichloromethane and water, and the organic phase is separated off, washed again with water and dried using magnesium sulfate. After filtration, the mixture is evaporated to dryness under reduced pressure. The residue is taken up in 80 ml of conc. hydrochloric acid and boiled on a reflux condenser overnight. The solution is cooled and extracted with 150 ml of ethyl acetate. The ethyl acetate phase is dried using magnesium sulfate and evaporated to dryness under reduced pressure. The residue is purified via a silica-gel column (eluent EA/PE 1:5, later EA), giving 3.6 g of 3-(4-fluoro-3-nitrophenyl)propionic acid.

d

4.0 g of 3-(4-fluoro-3-nitrophenyl)propionic acid is stirred at room temperature for 2 hours in 50 ml of dichloromethane containing 4 ml of thionyl chloride, then evaporated to dryness under reduced pressure. 10 g of Wang resin are suspended in 250 ml of dichloromethane and 3.23 ml of N-diisopropylethylamine. The acid chloride is added dropwise with cooling in an ice bath. The mixture is then stirred at room temperature for 24 hours. The reaction solution is filtered, and the solid phase is washed with 200 ml of each of dichloromethane, dimethylformamide (DMF), DMF/water, DMF, dichloromethane and methanol and dried under reduced pressure, giving 12.4 g of polymer-bound 3-(4-fluoro-3-nitrophenyl)propionic acid.

e

0.5 g of polymer-bound 3-(4-fluoro-3-nitrophenyl)propionic acid are suspended in 5 ml of DMF and stirred overnight at room temperature with 1.21 g of 4-methoxybenzylamine and 1.92 ml of N-diisopropylethylamine. The reaction solution is filtered, and the solid phase is washed with dichloromethane, dimethylformamide (DMF), DMF/water, DMF, dichloromethane and methanol and dried under reduced pressure, giving 0.52 g of polymer-bound 3-(4-methoxybenzylamino-3-nitrophenyl)propionic acid.

f

0.5 g of polymer-bound 3-(4-methoxybenzylamino-3-nitrophenyl)propionic acid are suspended in 4 ml of DMF and 1 ml of ethanol, and 2.55 g of tin(II) chloride dihydrate are added. The mixture is stirred overnight at 50° C. The reaction mixture is then filtered, and the filter cake is washed 3× with DMF, 2× with DMF/water (1:1), 3× with DMF, 3× with dichloromethane and 3× with methanol, giving 0.5 g of polymer-bound 3-(3-amino-4-benzylamino)propionic acid.

g

0.4 g of polymer-bound 3-(3-amino-4-methoxybenzylamino-)propionic acid are suspended in 4 ml of DMF and 2 ml of ethanol, and 0.48 g of cyanogen bromide is added. The mixture is stirred overnight at room temperature. The reaction mixture is then filtered, and the filter cake is washed 3× with DMF, 2× with DMF/water (1:1), 3× with DMF, 3× with dichloromethane and 3× with methanol, giving 0.4 g of polymer-bound 3-[2-amino-1-(4-methoxybenzyl)-1H-benzimidazol-5-yl]propionic acid.

h

0.2 g of polymer-bound 3-(2-amino-1-4-methoxy-1H-benzimidazol-5-yl)propionic acid are stirred for 30 minutes in 2 ml of trifluoroacetic acid/dichloromethane (1:1). The cleavage solution is evaporated under reduced pressure. The crude product obtained in this way is purified by means of preparative HPLC via an RP-18 column, giving 18 mg of 3-[2-amino-1-(4-methoxybenzyl)-1H-benzimidazol-5-yl]propionic acid (A).

i

3 ml of toluene are added to 0.4 g of polymer-bound 3-[2-amino-1-(4-methoxybenzyl)-1H-benzimidazol-5-yl]propionic acid, and the mixture is cooled in an ice bath under an argon atmosphere.

3.0 ml of diisobutylaluminium hydride (20% in toluene) are added, and the mixture is left to stand overnight at room temperature. The solid phase is filtered off and washed: 2× with toluene, 2× with THF, 1× with water/THF(1:1) and 2× with methanol.

1 molar HCl is then added to the combined organic phases until the precipitate formed dissolves again. The solution is diluted with a little water and washed 4 times with dichloromethane. The combined organic phases are dried over magnesium sulfate, filtered and evaporated to dryness. Purification by preparative HPLC gives 18.5 mg of 3-[2-amino-1-(4-methoxybenzyl)-1H-benzimidazol-5-yl]propan-1-ol (B).

### EXAMPLE 2

### Synthesis of 1-benzyl-5-morpholin-4-ylmethyl-1H-benzimidazol-2-ylamine

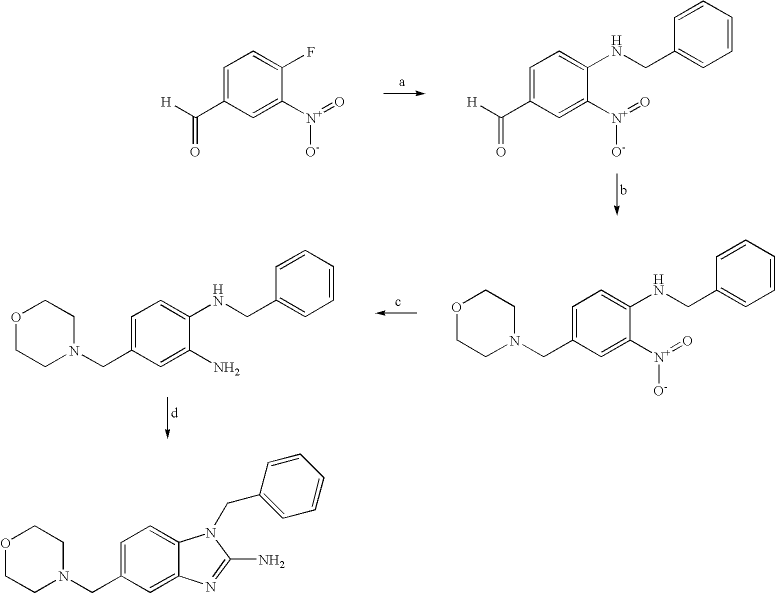

a

0.2 g of 4-fluoro-3-nitrobenzaldehyde and 30 ml of benzylamine are dissolved in DMF and stirred overnight at room temperature. The reaction mixture is then evaporated under reduced pressure and purified via a silica-gel column, giving 15 g of 4-benzylamino-3-nitrobenzaldehyde.

b

0.77 g of 4-benzylamino-3-nitrobenzaldehyde and 1.05 ml of morpholine are dissolved in 8 ml of absolute methanol. A solution of 0.19 g of sodium cyanoborohydride and 0.2 g of zinc(II) chloride in 5 ml of absolute methanol is added. The mixture is stirred at room temperature for 2 hours. 20 ml of 0.1M NaOH are then added, and the methanol is evaporated under reduced pressure. The aqueous phase is extracted with ethyl acetate (3 times 30 ml). The combined organic phases are extracted with water and saturated sodium chloride solution, dried using MgSO4, filtered and evaporated under reduced pressure. The crude product (0.8 g) was purified via a silica-gel column (eluent: PE/EA 1:1), giving 0.6 g of benzyl-(4-morpholin-4-yl-methyl-2-nitrophenyl)amine.

c

0.57 g of benzyl-(4-morpholin-4-ylmethyl-2-nitrophenyl)amine are dissolved in ethyl acetate, and 2.03 g of tin(II) chloride dihydrate are added. The mixture is boiled on a reflux condenser overnight. The solution was adjusted to pH 9 using 2.5 M NaOH and extracted with ethyl acetate. The organic phase was dried using magnesium sulfate, filtered and evaporated under reduced pressure. The crude product (0.5 g) was purified via a silica-gel column (eluent: PE/EA 1:3), giving 60 mg of N1-benzyl-4-morpholin-4-yl-methylphenyl-1,2-diamine.

d

60 mg of N1-benzyl-4-morpholin-4-ylmethylphenyl-1,2-diamine and 64.2 mg of cyanogen bromide are dissolved in 10 ml of ethanol/DMF 1:2 and stirred overnight at room temperature. The organic phase is evaporated under reduced pressure. The crude product was purified via a silica-gel column (eluent: PE/EA 1:3), giving 9.3 mg of 1-benzyl-5-morpholin-4-ylmethyl-1H-benzimidazol-2-ylamine.

The following compounds are obtained analogously:

| | MOLECULAR | Retention |
|---|---|---|
| COMPOUNDS | WEIGHT | time [min] |
| 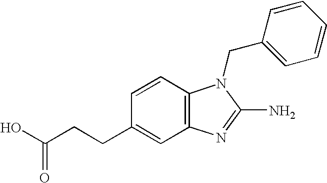 | 295.3 | 2.45 |
| 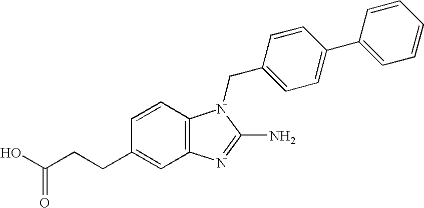 | 371.4 | 3.33 |
| 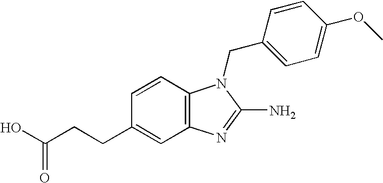 | 325.4 | 2.67 |
| 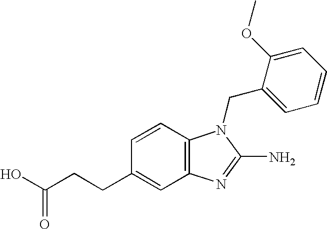 | 325.4 | 3.31 |
| 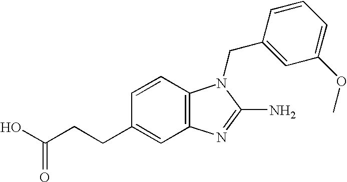 | 325.4 | 2.67 |
| 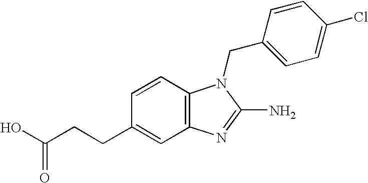 | 329.8 | 2.91 |
| 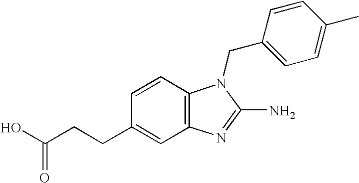 | 309.4 | 2.80 |
| 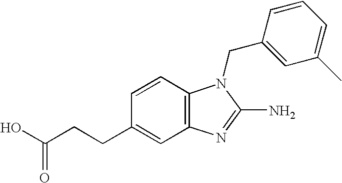 | 309.4 | 2.85 |
| 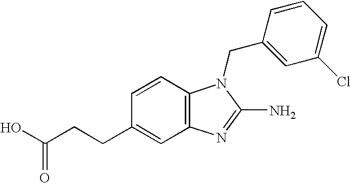 | 329.8 | 2.85 |
| 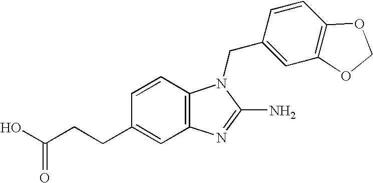 | 339.3 | 2.64 |
| 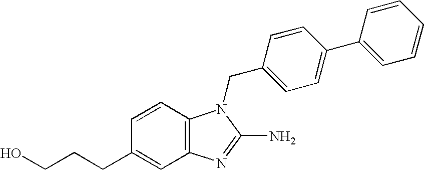 | 357.5 | 3.25 |
| 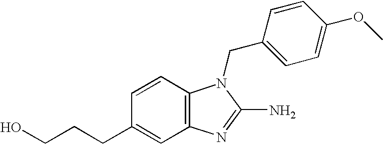 | 311.4 | 2.59 |
| 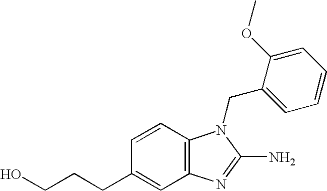 | 311.4 | 2.72 |
| 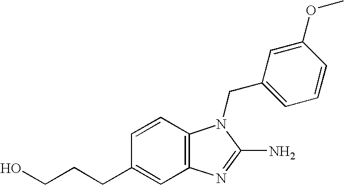 | 311.4 | 2.67 |
| 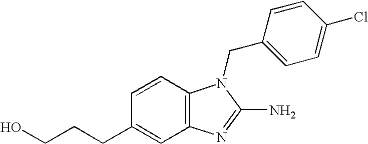 | 315.8 | 2.88 |
| 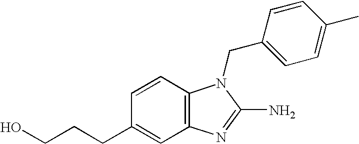 | 295.4 | 2.8 |
| 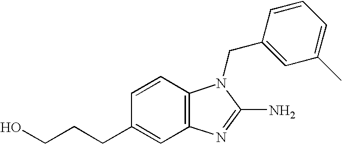 | 295.4 | 2.75 |
| 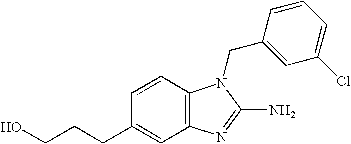 | 315.8 | 2.88 |
| 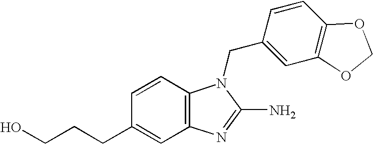 | 325.4 | 2.61 |
| 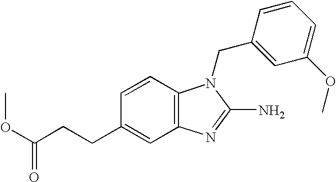 | 339.4 | 3.04 |
| 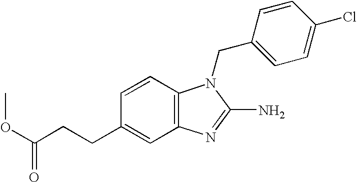 | 343.8 | 3.28 |
| 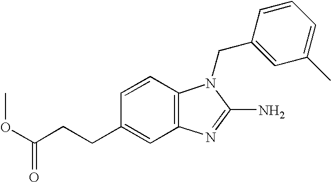 | 323.4 | 3.17 |
| 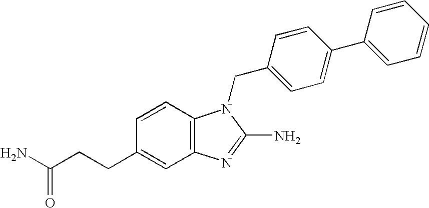 | 370.5 | 3.09 |
| 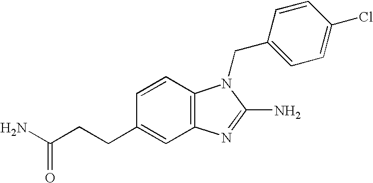 | 328.8 | 2.67 |
| 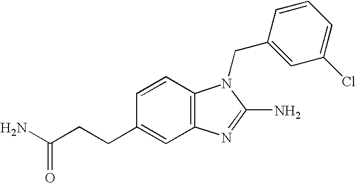 | 328.8 | 2.61 |
| 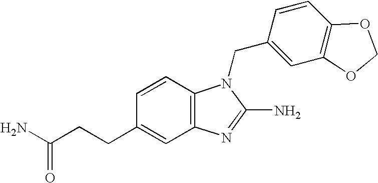 | 338.4 | 2.35 |
| 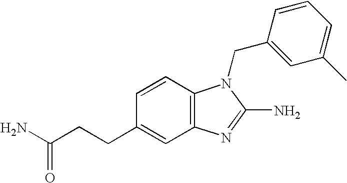 | 308.4 | 2.59 |
| 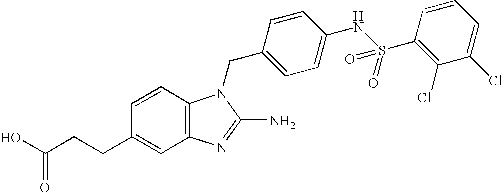 | 519.4 | 3.12 |
| 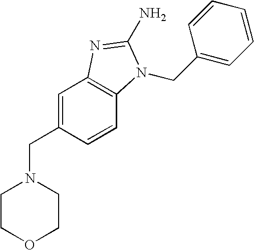 | 322.4 | 2.08 |
| 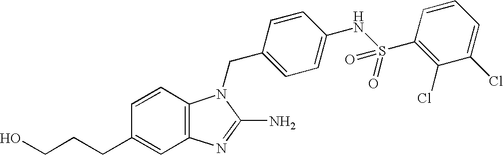 | 505.4 | 3.2 |
| 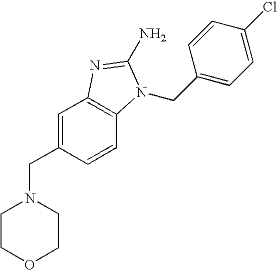 | 356.9 | 2.24 |
| 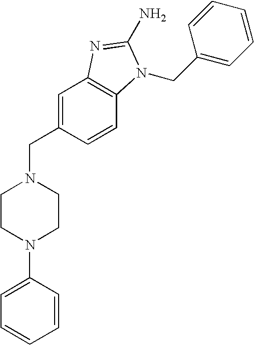 | 397.5 | 2.61 |
| 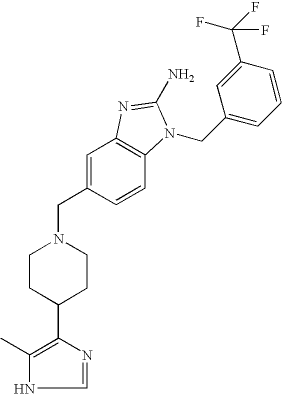 | 468.5 | 2.27 |
| 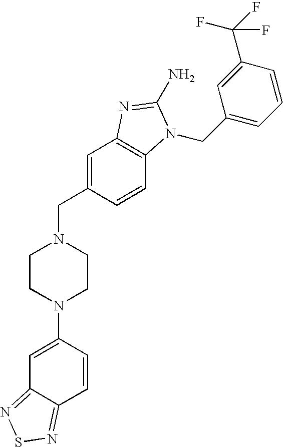 | 523.6 | 2.96 |
| 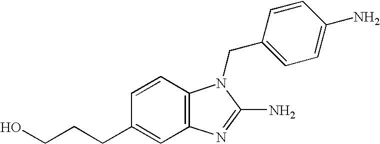 | 296.4 | 1.6 |
| 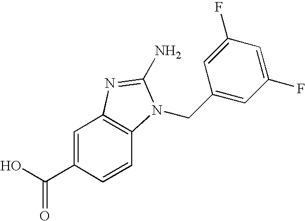 | 303.3 | 2.51 |
| 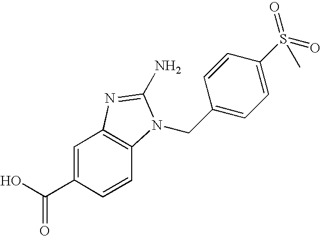 | 345.4 | 1.92 |
| 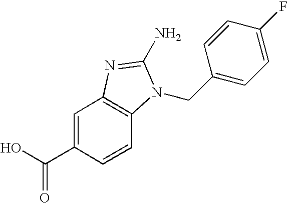 | 285.3 | 2.43 |

Pharmacological test results

| | Inhibition of TIE-2 |
|---|---|
| COMPOUND | IC₅₀ (nmol) |
| N-[4-(2-amino-5-bromo-benzimidazol-1-ylmethyl)- | 2780 |
| phenyl]-2,3-dichloro-benzenesulfonamide | |
| N-{4-[2-amino-5-(3-hydroxy-propyl)benzimidazol- | 320 |
| 1-yl-methyl]phenyl}-2,3-dichloro- | |
| benzenesulfonamide | |

The following examples relate to pharmaceutical compositions:

### EXAMPLE A

### Injection Vials

A solution of 100 g of an active ingredient according to the invention and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient according to the invention is melted with 100 g of soya lecithin and 1400 g of cocoa butter, poured into moulds and allowed to cool. Each suppository contains 20 mg of active ingredient.

### EXAMPLE C

### Solution

A solution is prepared from 1 g of an active ingredient according to the invention, 9.38 g of NaH₂PO₄.2 H₂O, 28.48 g of Na₂HPO₄.12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient according to the invention are mixed with 99.5 g of Vaseline under aseptic conditions.

### EXAMPLE E

### Tablets

A mixture of 1 kg of active ingredient, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient are introduced into hard gelatine capsules in a conventional manner in such a way that each capsule contains 20 mg of the active ingredient.

### EXAMPLE H

### Ampoules

A solution of 1 kg of an active ingredient according to the invention in 60 l of bidistilled water is sterile filtered, transferred into ampoules, lyophilised under sterile conditions and sealed under sterile conditions. Each ampoule contains 10 mg of active ingredient.

1. Compounds of the formula I 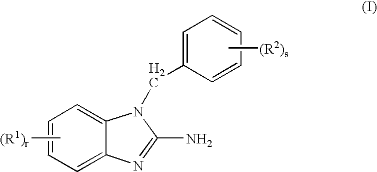
in which
R¹, R² each, independently of one another, denote R, Hal, CN, NO₂, NHR, NR₂, NHCOR, NHSO₂R, OR, COR, CONHR, SCF₃, SO₃R, SO₂R, SO₂NR₂, SR, COOH or COOA, where two radicals R² together may also be —O—CH₂—O— or —O—CH₂—CH₂—O—,
R denotes H, A, Ar, Het, (CH₂)ₚAr, or (CH₂)ₚHet,
p denotes 1, 2 or 3,
Ar denotes phenyl or naphthyl, each of which is unsubstituted or mono-, di- or trisubstituted by A, Hal, OH, OA, CN, NO₂, NH₂, NHA, NA₂, NHCOA, SCF₃, SO₂A, COOH, COOA, CONH₂, CONHA, CONA₂, NHSO₂A, SO₂NH₂, SO₂NHA, SO₂NA₂, CHO or COA,
A denotes unbranched, branched or cyclic alkyl having 1-10 C atoms, in which one or two CH₂ groups may be replaced by O or S atoms and/or by —CH═CH— groups and/or in addition 1-7 H atoms may be replaced by F and/or Cl and where A may be mono-, di- or trisubstituted by COOH, OH, COOA′ or CONH₂,
Het denotes a mono- or bicyclic saturated, unsaturated or aromatic heterocycle having 1 to 4 N, O and/or S atoms, which is unsubstituted, or may be mono-, di- or trisubstituted by carbonyl oxygen, Hal, A, —(CH₂)ₙ—Ar, —(CH₂)ₙ-cycloalkyl, OH, OA, NH₂, NHA, NA₂, NO₂, CN, COOH, COOA, CONH₂, CONHA, CONA₂, NHCOA, NHCONH₂, NHSO₂A, CHO, COA, SO₂NH₂ and/or S(O)ₘA,
A′ denotes unbranched, branched or cyclic alkyl having 1-6 C atoms,
m denotes 0, 1 or 2,
n denotes 0, 1, 2, 3 or 4,
Hal denotes F, Cl, Br or I,
r denotes 0, 1, 2, 3 or 4,
s denotes 0, 1, 2, 3, 4 or 5,
and pharmaceutically usable derivatives, salts, solvates and stereoisomers thereof, including mixtures thereof in all ratios.

2. Compounds according to claim 1 in which
R¹ denotes R, COOH or COOA,
and pharmaceutically usable derivatives, salts, solvates and stereoisomers thereof, including mixtures thereof in all ratios.

3. Compounds according to claim 1 in which
R² denotes R, OR, NH₂, Hal, SO₂A or NHSO₂R, where two radicals R² together may also be —O—CH₂—O— or —O—CH₂—CH₂—O—,
and pharmaceutically usable derivatives, salts, solvates and stereoisomers thereof, including mixtures thereof in all ratios.

4. Compounds according to claim 1 in which
Ar denotes phenyl which is unsubstituted or mono-, di- or trisubstituted by Hal,
and pharmaceutically usable derivatives, salts, solvates and stereoisomers thereof, including mixtures thereof in all ratios.

5. Compounds according to claim 1 in which
A denotes unbranched, branched or cyclic alkyl having 1, 2, 3, 4, 5 or 6 C atoms, in which, in addition, 1-7 H atoms may be replaced by F and/or Cl, where A may also be mono-, di- or trisubstituted by COOH, OH, COOA′ or CONH₂,
and pharmaceutically usable derivatives, salts, solvates and stereoisomers thereof, including mixtures thereof in all ratios.

6. Compounds according to claim 1 in which
R¹ denotes R, COOH or COOA,
R² denotes R, OR, NH₂, Hal, SO₂A or NHSO₂R, where two radicals R² together may also be —O—CH₂—O— or —O—CH₂—CH₂—O—,
R denotes H, A, Ar, Het, (CH₂)ₚAr, or (CH₂)ₚHet,
p denotes 1, 2 or 3,
Ar denotes phenyl or naphthyl, each of which is unsubstituted or mono-, di- or trisubstituted by A, Hal, OH, OA, CN, NO₂, NH₂, NHA, NA₂, NHCOA, SCF₃, SO₂A, COOH, COOA, CONH₂, CONHA, CONA₂, NHSO₂A, SO₂NH₂, SO₂NHA, SO₂NA₂, CHO, COA,
A denotes unbranched, branched or cyclic alkyl having 1-10 C atoms, in which one or two CH₂ groups may be replaced by O or S atoms and/or by —CH═CH— groups and/or in addition 1-7 H atoms may be replaced by F and/or Cl, where A may be mono-, di- or trisubstituted by COOH, OH, COOA′ or CONH₂,
A′ denotes an unbranched, branched or cyclic alkyl having 1-6 C atoms,
Hal denotes F, Cl, Br, or I,
r denotes 0, 1, 2, 3 or 4,
s denotes 0, 1, 2, 3, 4 or 5,
and pharmaceutically usable derivatives, salts, solvates and stereoisomers thereof, including mixtures thereof in all ratios.

7. Compounds according to claim 1 in which
R¹ denotes A, (CH₂)ₚHet, COOH, or COOA,
Het denotes a mono- or bicyclic saturated, unsaturated or aromatic heterocycle having 1 to 4 N, O and/or S atoms, which is unsubstituted, or may be mono-, di- or trisubstituted by carbonyl oxygen, Hal, A, —(CH₂)ₙ—Ar, —(CH₂)ₙ-cycloalkyl, OH, OA, NH₂, NHA, NA₂, NO₂, CN, COOH, COOA, CONH₂, CONHA, CONA₂, NHCOA, NHCONH₂, NHSO₂A, CHO, COA, SO₂NH₂ and/or S(O)ₘA,
and pharmaceutically usable derivatives, salts, solvates and stereoisomers thereof, including mixtures thereof in all ratios.

8. Compounds according to claim 1 in which
R² can be Ar, OA, Hal, A, NHSO₂Ar, NH₂, SO₂A, where two radicals R² together may also be —O—CH₂—O— or —O—CH₂—CH₂—O—,
and pharmaceutically usable derivatives, salts, solvates and stereoisomers thereof, including mixtures thereof in all ratios.

9. Process for the preparation of compounds of the formula I according to claim 1 and pharmaceutically usable derivatives, solvates and stereoisomers thereof, characterised in that a compound of the formula II 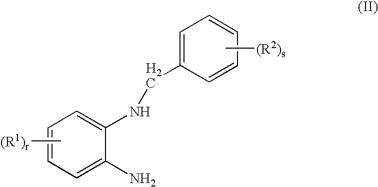
in which R¹, R², r and s have the meaning indicated in claim 1, is reacted with BrCN
and/or a base or acid of the formula I are converted into one of its salts.

10. Compounds according to claim 1, selected from the group
3-(2-amino-1-benzyl-1H-benzimidazol-5-yl)propionic acid,
3-(2-amino-1-biphenyl-4-ylmethyl-1H-benzimidazol-5-yl)propionic acid,
3-[2-amino-1-(4-methoxybenzyl)-1H-benzimidazol-5-yl]propionic acid,
3-[2-amino-1-(2-methoxybenzyl)-1H-benzimidazol-5-yl]propionic acid,
3-[2-amino-1-(3-methoxybenzyl)-1H-benzimidazol-5-yl]propionic acid,
3-[2-amino-1-(4-chlorobenzyl)-1H-benzimidazol-5-yl]propionic acid,
3-[2-amino-1-(4-methylbenzyl)-1H-benzimidazol-5-yl]propionic acid,
3-[2-amino-1-(3-methylbenzyl)-1H-benzimidazol-5-yl]propionic acid,
3-[2-amino-1-(3-chlorobenzyl)-1H-benzimidazol-5-yl]propionic acid,
3-(2-amino-1-benzo-1,3-dioxol-5-ylmethyl-1H-benzimidazol-5-yl)propionic acid,
3-(2-amino-1-biphenyl-4-ylmethyl-1H-benzimidazol-5-yl)propan-1-ol,
3-[2-amino-1-(4-methoxybenzyl)-1H-benzimidazol-5-yl]propan-1-ol,
3-[2-amino-1-(2-methoxybenzyl)-1H-benzimidazol-5-yl]propan-1-ol,
3-[2-amino-1-(3-methoxybenzyl)-1H-benzimidazol-5-yl]propan-1-ol,
3-[2-amino-1-(4-chlorobenzyl)-1H-benzimidazol-5-yl]propan-1-ol,
3-[2-amino-1-(4-methylbenzyl)-1H-benzimidazol-5-yl]propan-1-ol,
3-[2-amino-1-(3-methylbenzyl)-1H-benzimidazol-5-yl]propan-1-ol,
3-[2-amino-1-(3-chlorobenzyl)-1H-benzimidazol-5-yl]propan-1-ol,
3-(2-amino-1-benzo-1,3-dioxol-5-ylmethyl-1H-benzimidazol-5-yl)propan-1-ol,
3-[2-amino-1-(3-methoxybenzyl)-1H-benzimidazol-5-yl]propionic acid methyl ester,
3-[2-amino-1-(4-chlorobenzyl)-1H-benzimidazol-5-yl]propionic acid methyl ester,
3-[2-amino-1-(3-methylbenzyl)-1H-benzimidazol-5-yl]propionic acid methyl ester,
3-(2-amino-1-biphenyl-4-ylmethyl-1H-benzimidazol-5-yl)propionamide,
3-[2-amino-1-(4-chlorobenzyl)-1H-benzimidazol-5-yl]propionamide
3-(2-amino-1-benzo-1,3-dioxol-5-ylmethyl-1H-benzimidazol-5-yl)propionamide,
3-[2-amino-1-(3-methylbenzyl)-1H-benzimidazol-5-yl]propionamide,
3-{2-amino-1-[4-(2,3-dichlorobenzenesulfonylamino)benzyl]-1H-benzimidazol-5-yl}propionic acid,
1-benzyl-5-morpholin-4-ylmethyl-1H-benzimidazol-2-ylamine,
N-{4-[2-amino-5-(3-hydroxypropyl)benzimidazol-1-ylmethyl]phenyl}-2,3-dichlorobenzenesulfonamide,
1-(4-chlorobenzyl)-5-morpholin-4-ylmethyl-1H-benzimidazol-2-ylamine,
1-benzyl-5-(4-phenylpiperazin-1-ylmethyl)-1H-benzimidazol-2-ylamine,
5-[4-(5-methyl-1H-imidazol-4-yl)piperidin-1-ylmethyl]-1-(3-trifluoromethylbenzyl)-1H-benzimidazol-2-ylamine,
5-(4-benzo-1,2,5-thiadiazol-5-ylpiperazin-1-ylmethyl)-1-(3-trifluoromethylbenzyl)-1H-benzimidazol-2-ylamine,
3-[2-amino-1-(4-aminobenzyl)-1H-benzimidazol-5-yl]propan-1-ol,
2-amino-1-(3,5-difluorobenzyl)-1H-benzimidazole-5-carboxylic acid,
2-amino-1-(4-methanesulfonylbenzyl)-1H-benzimidazole-5-carboxylic acid,
2-amino-1-(4-fluorobenzyl)-1H-benzimidazole-5-carboxylic acid,
and pharmaceutically usable derivatives, solvates and stereoisomers thereof, including mixtures thereof in all ratios.

11. Medicament comprising at least one compound according to claim 1 and/or pharmaceutically usable derivatives, solvates and stereoisomers thereof, including mixtures thereof in all ratios, and optionally excipients and/or adjuvants, as tyrosine kinase modulator.

12. Use of compounds according to claim 1,
and pharmaceutically usable derivatives, solvates and stereoisomers thereof, including mixtures thereof in all ratios, for the preparation of a medicament for the treatment and/or prophylaxis of diseases in which the inhibition, regulation and/or modulation of kinase signal transduction plays a role.

13. Use according to claim 12, where the kinases are selected from the group of tyrosine kinases.

14. Use according to claim 13, where the tyrosine kinases are TIE-2, VEGFR, PDGFR, FGFR and/or FLT/KDR.

15. Use according to claim 13 of compounds of the invention, and pharmaceutically usable derivatives, solvates and stereoisomers thereof, including mixtures thereof in all ratios, for the preparation of a medicament for the treatment of diseases which are influenced by inhibition of tyrosine kinases by the compounds.

16. Use according to claim 14 for the preparation of a medicament for the treatment of diseases which are influenced by inhibition of TIE-2, VEGFR, PDGFR, FGFR and/or FLT by the compounds.

17. Use according to claim 15, where the disease to be treated is a tumour.

18. Use according to claim 17, where the tumour a solid tumour from the group of tumours of the squamous epithelium, bladder, stomach, kidneys, head and neck, oesophagus, cervix, thyroid, intestine, liver, brain, prostate, urogenital tract, lymphatic system, larynx and/or lung.

19. Use according to claim 17, where the solid tumour originates from the group of lung adenocarcinoma, small cell lung carcinomas, pancreatic cancer, glioblastomas, colon carcinoma and breast carcinoma.

20. Use according to claim 15, where the disease to be treated is a tumour of the blood and immune system.

21. Use according to claim 20, where the tumour originates from the group of acute myeloid leukaemia, chronic myeloid leukaemia, acute lymphatic leukaemia and/or chronic lymphatic leukaemia.

22. Use according to claim 15 for the treatment of a disease in which angiogenesis is implicated.

23. Use according to claim 22, where the disease is an eye disease.

24. Use according to claim 23 for the treatment of retinal vascularisation, diabetic retinopathy and/or age-induced macular degeneration.

25. Use according to claim 15 for the treatment of bone pathologies, where the bone pathology originates from the group osteosarcoma, osteoarthritis and rickets.

26. Medicament comprising at least one compound according to claim 1 and/or pharmaceutically usable derivatives, solvates and stereoisomers thereof, including mixtures thereof in all ratios, and at least one further medicament active ingredient.

27. Set (kit) consisting of separate packs of
(a) an effective amount of a compound according to claim 1 and/or pharmaceutically usable derivatives, solvates and stereoisomers thereof, including mixtures thereof in all ratios,
and
(b) an effective amount of a further medicament active ingredient.

28. Use of compounds according to claim 1 and/or physiologically acceptable salts and solvates thereof for the preparation of a medicament for the treatment of solid tumours, where a therapeutically effective amount of a compound according to claim 1 is administered in combination with a compound from the group 1) oestrogen receptor modulator, 2) androgen receptor modulator, 3) retinoid receptor modulator, 4) cytotoxic agent, 5) antiproliferative agent, 6) prenyl-protein transferase inhibitor, 7) HMG-CoA reductase inhibitor, 8) HIV protease inhibitor, 9) reverse transcriptase inhibitor and 10) other angiogenesis inhibitors.

29. Use of compounds according to claim 1 and/or physiologically acceptable salts and solvates thereof for the preparation of a medicament for the treatment of solid tumours, where a therapeutically effective amount of a compound according to claim 1 is administered in combination with radiotherapy and a compound from the group 1) oestrogen receptor modulator, 2) androgen receptor modulator, 3) retinoid receptor modulator, 4) cytotoxic agent, 5) antiproliferative agent, 6) prenyl-protein transferase inhibitor, 7) HMG-CoA reductase inhibitor, 8) HIV protease inhibitor, 9) reverse transcriptase inhibitor and 10) other angiogenesis inhibitors.

30. Use according to claim 12 for the preparation of a medicament for the treatment of diseases which are based on disturbed TIE-2 activity,
where a therapeutically effective amount of a compound of the invention is administered in combination with a growth-factor receptor inhibitor.

31. Use according to claim 12 for the treatment of diseases are selected from the group of hyperproliferative and non-hyperproliferative diseases which do not belong to the cancerous diseases.

32. Use according to claim 31, where the non-cancerous diseases are selected from the group consisting of psoriasis, arthritis, inflammation, contact dermatitis and delayed hypersensitivity reaction, endometriosis, scarring, benign prostatic hyperplasia, immunological diseases, autoimmune diseases and immunodeficiency diseases.
